(19) United States
(12) Patent Application Publication
(10) Pub. No.: US 2021/0015451 A1
(43) Pub. Date: Jan. 21, 2021

(54) METHODS FOR VAGINAL TACTILE AND ULTRASOUND IMAGE FUSION

(71) Applicant: Advanced Tactile Imaging, Inc., Trenton, NJ (US)

(72) Inventor: Vladimir Egorov, Princeton, NJ (US)

(21) Appl. No.: 17/028,636

(22) Filed: Sep. 22, 2020

Related U.S. Application Data
(63) Continuation-in-part of application No. 15/249,672, filed on Aug. 29, 2016, now abandoned.
Application No. 15/249,672 is a continuation-in-part of application No. 16/055,265, filed on Aug. 6, 2018.
(60) Provisional application No. 62/215,227, filed on Sep. 8, 2015.

(51) Int. Cl. A61B 8/12; A61B 8/08

(52) U.S. Cl. CPC A61B 8/12; A61B 8/08; A61B 8/485; A61B 8/5246

(57) ABSTRACT

A comprehensive evaluation of the female pelvic floor tissues and structures is facilitated by a method of image fusion of two different imaging modalities, a tactile response data or tactile image is described as fused with ultrasound image. Both tactile response and ultrasound images may be acquired at the same time using a vaginal probe equipped with adjacent arrays of tactile sensors and ultrasound transducer elements.

### CROSS-REFERENCE TO RELATED APPLICATIONS

This US patent application is a continuation-in-part of the U.S. patent application Ser. No. 15/249,672 filled Aug. 29, 2016, by the same inventor with the title “METHODS AND PROBES FOR VAGINAL TACTILE AND ULTRASOUND IMAGING”, which claims a priority benefit from the U.S. Provisional Patent Application No. 62/215,227 filed Sep. 8, 2015, by the same inventor with the same title. This patent application is also a continuation-in-part of the U.S. patent application Ser. No. 16/055,265 filled Aug. 6, 2018, by the same inventor with the title “METHODS FOR BIOMECHANICAL MAPPING OF THE FEMALE PELVIC FLOOR”. This patent application further claims a priority date benefit of the U.S. Provisional Patent Application No. 62/706,663 filed Sep. 2, 2020, by the same inventor with the title “METHODS FOR VAGINAL TACTILE AND ULTRASOUND IMAGE FUSION”. All of the above documents are incorporated herein in their respective entireties by reference for all purposes.

### GOVERNMENT-SPONSORED RESEARCH

This invention was made with the US Government support under grant No. R43HD097805 awarded by Eunice Kennedy Shriver National Institute of Child Health & Human Development, USA. The Government has certain rights in this invention.

### FIELD OF THE INVENTION

The present invention generally relates to female pelvic floor imaging. Specifically, the invention describes methods and devices for providing vaginal tactile and ultrasound imaging, and image fusion techniques to characterize the anatomical, biomechanical and functional conditions of the female pelvic floor.

### BACKGROUND

Pelvic organ prolapse (POP) is an abnormal descent or herniation of the pelvic organs from their normal attachment sites or their normal position in the pelvis. This condition is often associated with concomitant pelvic floor disorders, including urinary and fecal incontinence, pelvic pain, sexual dysfunction, voiding dysfunction, and social isolation. A recent study provided a projection that by the year 2050, 43.8 million women, or nearly 33% of the adult female population in the US, would be affected by at least one troublesome pelvic floor disorder. The lifetime risk of undergoing surgery for pelvic organ prolapse or urinary incontinence is near 20%.

Urinary incontinence (UI) is a storage symptom and defined as the complaint of any involuntary loss of urine. The most common type of UI is stress urinary incontinence (SUI), defined as the complaint of involuntary leakage on effort or exertion, or on sneezing or coughing. Estimates of the prevalence of this disorder vary depending on the affected population and the definition of UI. Using the inclusive definition of any leakage occurring at least once in the past year, the prevalence of UI ranges from 25% to 51%.

The female pelvic floor comprises the pelvic diaphragm muscles (pubococcygeus, puborectalis, and iliococcygeus, together known as the levator ani), the urogenital diaphragm muscles (ischiocavernosus, bulbospongiosus, and transverses perinei superficialis, together known as the perineal muscles); and the urethral and anal sphincter muscles. These muscles interrelate with each other both anatomically and functionally. The normal action of the pelvic floor muscles (PFM) has been described as a squeeze around the pelvic openings and an inward lift. The pelvic floor disorders result from the neuro-urinary pathology as well as muscle functional impairment due to changes in biomechanical properties of soft tissues associated with age. The anatomy of the pelvic floor is complex and clinical examination alone is often insufficient to diagnose and assess the pathology. A large number of these patients suffer for many years without relief—often due to the lack of objective findings necessary to plan proper treatment. Because abnormalities of the different pelvic compartments are frequently interrelated, their thorough diagnostic characterization is paramount for proper disease management and treatment. That is why pelvic floor muscle's characterization and diagnosis must include dynamic/functional stress-and-strain imaging to allow biomechanical (force) measurements. The need exists for a comprehensive imaging solution including the representation of different imaging modalities in order to improve the characterization of the pelvic tissues.

Treatment options for POP and SUI include surgery and pelvic muscle training. An invasive surgical approach is considered the ultimate treatment for both POP and SUI. It was found that surgical treatments of recurrent SUI are associated with high failure rates. Physical training and medications are often not effective. There is a need for the objective and quantitative pre- and post-surgery assessment of pelvic floor conditions to improve evidence-based management in urogynecologic surgeries. There is also a need for identifying the most effective and durable repair with the lowest morbidity rate.

Tactile Imaging is a medical imaging modality that translates the sense of touch into a digital image. The tactile image is a function of P(x,y,z), where P is the pressure on soft tissue surface under applied deformation, and x,y,z are coordinates where pressure P was measured. Functional Tactile Imaging translates muscle activity into dynamic pressure pattern P(x,y,t) for an area of interest, where t is time and x,y are coordinates where pressure P was measured. Muscle activity may include a variety of types such as muscle voluntary contraction, involuntary reflex contraction, involuntary relaxation, specific maneuvers (e.g Valsalva maneuver) [Egorov, et al. Biomechanical mapping of the female pelvic floor: changes with age, parity, and weight. Pelviperineology 2019; 38: 3-11]. Tactile imaging devices and systems were developed to provide an alternative imaging modality for breast, prostate, and vagina tissues, as described in U.S. Pat. Nos. 6,620,115; 6,142,959; and 8,187,208.

Although tactile imaging is a simple and attractive technique to characterize soft tissues, it may not by itself provide the comprehensive information needed for proper diagnosis of POP or UI. Ultrasound imaging is another widely used technique for soft tissue imaging. Using these two imaging techniques independently and consecutively makes it difficult for a clinician to assess the entire situation with a particular subject as both measurements are not done at the same time and for the same area of interest in the female pelvic floor.

There is a need to conduct the entire evaluation of the pelvic tissues at the same time and provide a clinician with a comprehensive tissue imaging report combining more than one imaging modality.

### SUMMARY

The object of the present invention is to overcome the drawbacks of the prior art and to provide novel methods for objective and comprehensive characterization of the female pelvic floor tissues.

Another object of the invention is to provide novel methods for objective characterization and real-time visualization of contractile capabilities of pelvic floor muscles.

A further object of the invention is to provide novel methods for simultaneous recording tactile images and ultrasound strain images for vaginal and pelvic floor tissues during vaginal wall deformations by a single vaginal probe.

Another object of the invention is to provide novel methods for objective visualization and real-time detection of damaged/weak pelvic floor muscles by recording a tactile response and ultrasound image changes during muscle contractions at the same time.

Another yet object of the invention is to provide novel methods for composing stress-strain images from tactile and ultrasound imaging.

A further object of the invention is to provide novel methods for composing muscle functional images from tactile responses and ultrasound images.

Another yet object of the invention is to provide novel methods for characterizing pelvic floor tissues and muscles as biomechanical elements based on tissue strain-stress images and muscle functional images.

According to the present invention, novel methods for characterization of female pelvic floor via vaginal tactile and ultrasound image fusion may include the following steps:
a) inserting a vaginal probe into vagina, the vaginal probe may be equipped with a plurality of tactile sensors and a plurality of ultrasound transducer elements, which may be positioned adjacent to and alongside with the plurality of tactile sensors,
b) simultaneously generating tactile images and ultrasound images of the female pelvic floor while deforming vaginal walls by moving the vaginal probe. Tactile images in this case may be generated using the output of the plurality of tactile sensors, while the ultrasound images may be generated using the output of the plurality of ultrasound transducer elements,
c) simultaneously generating dynamic tactile patterns and generating ultrasound images of the female pelvic floor during pelvic muscle contractions. The dynamic tactile patterns may be generated using outputs of the same plurality of tactile sensors,
d) producing image fusion of the tactile images and the ultrasound images generated in step (b) for characterization of anatomical and biomechanical conditions of the female pelvic floor, and
e) producing image fusion of the dynamic tactile patterns and the ultrasound images generated in step (c) for characterization of anatomical and functional conditions of the female pelvic floor.

### BRIEF DESCRIPTION OF DRAWINGS

The patent or application file contains at least one drawing executed in color. Copies of this patent or patent application publication with color drawing(s) will be provided by the Office upon request and payment of the necessary fee.

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 illustrates the vaginal probe location after performing examination via probe insertion (Test 1). Tests 2-5, 7 and 8 are performed for such probe location recording tactile responses from anterior and posterior vaginal walls and ultrasound images for the vaginal compartments,

FIG. 2 presents one embodiment of a probe configured for vaginal tactile and ultrasound imaging,

FIG. 3a illustrates probe insertion for Test 1,

FIG. 3b is an exemplary acquired tactile image for anterior and posterior compartments for Test 1,

FIG. 4a illustrates probe elevation for Test 2,

FIG. 4b shows an exemplary acquired tactile image for anterior and posterior compartments for Test 2,

FIG. 5a illustrates probe rotation for Test 3,

FIG. 5b shows an exemplary acquired tactile image for the left and right sides of the vagina in Test 3,

FIG. 6 presents a flow chart of tactile and ultrasound data acquisition for the female pelvic floor,

FIG. 7 illustrates tactile and ultrasound image fusion for probe insertion data for Test 1 to facilitate elasticity assessment of critical structures for a first example of a 66 y.o. woman with normal pelvic floor conditions,

FIG. 8 illustrates tactile and ultrasound image fusion for Test 2 data (probe elevation) which allows assessment of pelvic floor support; normal pelvic floor conditions (left panel, A1-A4), and pelvic organ prolapse stage II (right panel, B1-B4),

FIG. 9 illustrates ultrasound image fusion with tactile responses for Test 4 data for the second example of a patient with anterior Stage III POP and overactive bladder at rest (left panel) and at Valsalva maneuver (right panel),

FIG. 10 illustrates ultrasound image fusion with tactile responses for Test 5 data for the third example of a patient without posterior prolapse, but with uterine Stage II POP at rest (left panel) and at voluntary pelvic muscle contraction (right panel),

FIG. 11 illustrates ultrasound image fusion with tactile responses for Test 8 data for the fourth example of a patient with normal pelvic support (no prolapse), but with stress urinary incontinence and hyperactive urethra,

FIG. 12 is a flow chart for an embodiment of a method for vaginal tactile and ultrasound imaging, and

FIG. 13 is a flow chart for an embodiment of a method for vaginal tactile and ultrasound image fusion.

### DETAILED DESCRIPTION OF THE INVENTION

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, components and/or circuits have not been described in detail in order to avoid unnecessarily obscuring claimed subject matter. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

Specific terms are used in the following description, which are defined as follows:

“tactile sensor” is a sensor capable to measure an applied force averaged per sensor area or pressure thereon and transform it into an electrical signal to be used in tactile image formation;

“ultrasound transducer element” is a transducer capable to emit and receive an acoustic wave and transform it into an electrical signal to be used in ultrasound image formation;

“stress” is a force per unit of area (pressure) measured at the surface of a vaginal wall (kPa);

“strain” is a soft tissue displacement under tissue deformation (mm);

“tissue elasticity” is a capability of a soft tissue to resist against the applied load at relatively small deformation (between 0 and 13 mm), which is calculated as a ratio of stress to strain (kPa/mm);

“pelvic floor support” is a capability of integrated pelvic structures in the posterior compartment to resist against the applied load at significant (above 13 mm and up to 45 mm) deformation, which is calculated as a ratio of stress to strain (kPa/mm);

“muscle function” is a capability of muscle to contract;

“muscle strength” is a capability of muscle to generate force or pressure change on vaginal wall during muscle contraction (N or kPa).

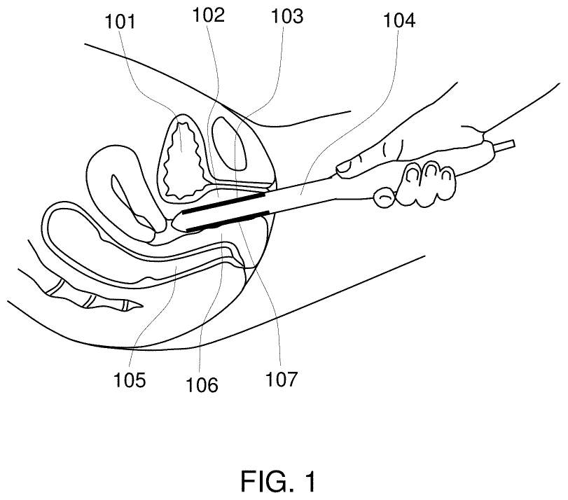

FIG. 1 shows a vaginal probe 104 in sagittal cross-section after its insertion into vagina. Bladder 101 and rectum 105 are shown for anatomical references. The same probe position may be used for recording of tactile images, dynamic tactile patterns (tactile responses), and ultrasound images from one side or both sides of the vaginal canal. Recording of the tactile images or tactile responses from two opposing vaginal walls contacting anterior 102 and posterior 106 compartments may be provided by means of two tactile sensor arrays consisting of a plurality of tactile sensors placed along the black lines 103 and 107 on both sides of the vaginal probe 104. Recording of ultrasound images may be provided by means of two ultrasound arrays consisting of a plurality of ultrasound elements placed along the same black lines 103 and 107 on both sides of the vaginal probe 104, or by means of a single ultrasound array consisting of a plurality of ultrasound elements placed along the black line 103 or 107. The tactile and ultrasound arrays allow recording of respective sensor output signals during tissue deformation by moving the vaginal probe 104 and/or during pelvic floor muscle contraction as well as pelvic floor muscles at rest. Tactile measurements at muscle rest provide data for muscle tone, which is considered an important characteristic of muscle conditions. The pelvic floor muscle contraction may take a variety of forms, including a voluntary muscle contraction for anterior versus posterior and left side versus right side, an involuntary (reflex) muscle contraction at cough, muscle contraction during Valsalva maneuver, and involuntary muscle relaxation.

To accurately record tactile and ultrasound images during muscle contraction, the vaginal probe 104 may be held in place without any displacements along the vaginal canal, ideally by keeping the vaginal probe 104 oriented in parallel to the vaginal canal during muscle contractions. The patient may be placed in a lithotomy position during the vaginal probe 104 insertion and imaging. Furthermore, the patient may be asked to contract (squeeze) pelvic floor muscles to enable recording of tactile and ultrasound signals on the flat rigid surfaces 103 and 107 of the vaginal probe 104. The patient may be also asked to follow specific instructions from a medical professional used in clinical practice for pelvic floor voluntary and involuntary (cough) muscle contraction as well as specific muscle maneuvers (Valsalva).

The portion of the vaginal probe 104 where the tactile and ultrasound sensors are located along the lines 103 and 107 may have at least one of rectangular, ellipsoidal, or circular cross-sectional shapes. In embodiments, the vaginal probe 104 may be shaped for atraumatic insertion into vagina and may have a tapered and generally rectangular cross-section with rounded edges and angles, so that smaller sides of the vaginal probe 104 may be equipped with at least some of the tactile sensors and ultrasound elements. A suitable lubricating gel may be used with the vaginal probe 104 insertion into vagina.

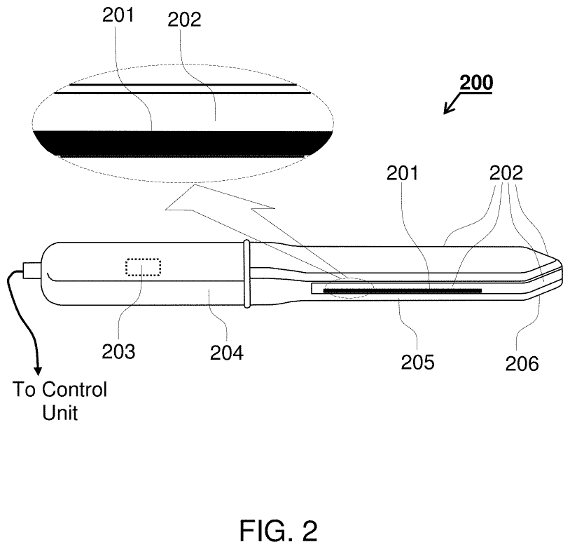

FIG. 2 presents an exemplary embodiment of a vaginal probe 200 for vaginal tactile and ultrasound imaging. The vaginal probe housing may have a handle 204 and an elongated portion with two parallel opposite sides 205 and a front tapered portion 206 at the vaginal probe end 204. The elongated portion of the vaginal probe housing may support a plurality of tactile sensors forming together a tactile array 202 located over at least a portion of the vaginal probe housing in a predefined relationship of tactile sensor positions to each other and to the vaginal probe housing. The tactile array 202 may be configured to record a tactile response from vaginal walls. The tactile array 202 may be subdivided into two arrays, one array per probe side.

The elongated portion of the vaginal probe housing may also house a plurality of ultrasound transducer elements forming together an ultrasound array 201 located over at least a portion of the vaginal probe housing in a predefined relationship of ultrasound element positions to each other and the vaginal probe housing. The ultrasound array 201 may be configured to acquire ultrasound images for soft tissues within a pelvic floor from one side of the vaginal probe or two opposite sides of the vaginal canal, e.g. anterior and posterior compartments.

Both the tactile array 202 sensors and the ultrasound array 201 transducer elements may be positioned adjacent to each other so that both respective tactile image and ultrasound image may be acquired at the same time and for the same portion of the pelvic tissues.

In embodiments, both the tactile array 202 and the ultrasound array 201 may be build from the same elements such as capacitive micromachined ultrasound transducers (CMUT) or tactile capacitive sensors so that both respective tactile image and ultrasound images may be acquired by the same transducers at the same time for the same portion of the pelvic tissues.

The vaginal probe 200 may be operatively connected to a control unit, which may be configured to provide a clinician with a control interface for operating the vaginal probe 200 as well as a suitable display for visualizing all signals and images acquired during the procedure.

Further referring to FIG. 2, the tactile array 202 and ultrasound array 201 may be suitably configured to acquire their respective tactile and ultrasound signals from a portion or the entire length of the vaginal canal. A certain minimum resolution may be required to be achieved by these imaging modalities. A linear resolution of at least 5 mm or better may be suitable for tactile sensors. For ultrasound sensors at least 1 mm resolution or better may be required. The tactile and ultrasound signal acquisition may be synchronized in time. The ultrasound transducer elements may accompany the tactile array along a part of or the entire length of the tactile array.

The vaginal probe 200 may be further equipped with an orientation sensor 203, which allows measurement of at least the probe elevation and rotation angles. The orientation sensor 203 may serve as a motion tracking system and may include at least one of an accelerometer, a magnetometer, or a gyroscope. Pressure signals from the tactile array 202 may be used to calculate an insertion depth of the vaginal probe into a vagina. Both the orientation angle and probe insertion depth may be used as part of a function of the motion tracking system, which provides determination of probe position relative to pelvic floor structures.

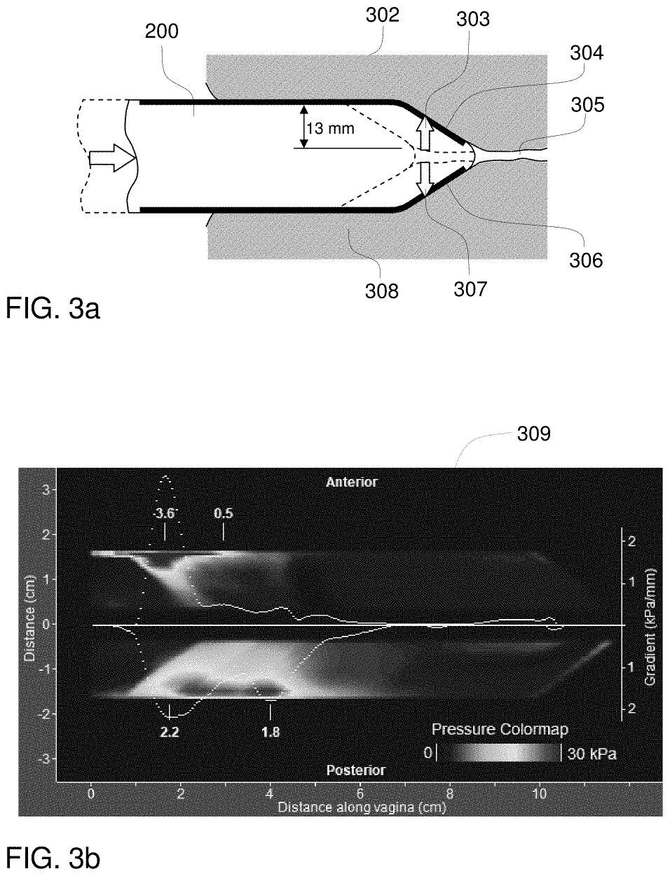

FIG. 3a illustrates the probe insertion (Test 1), while FIG. 3b shows an example of an acquired tactile image 309 for anterior 302 and posterior 308 vaginal compartments. The vaginal probe 200 may be designed to deform the vaginal walls in an orthogonal direction and away from each other as shown by arrows 303 and 307 from the vaginal canal 305 during the vaginal probe insertion. The linear motion of the vaginal probe, in this case, may be translated into vaginal tissue deformation in a definitive manner, as only the pressure sensors on the angled probe tip 304 and 306 may be used to create the tactile image 309. These sensors 304 and 307 may be configured to measure the tactile feedback from the vaginal walls for up to about 13 mm deformation per side from the vaginal canal 305. During the clinical examination, before the vaginal probe insertion, the anterior and posterior vaginal walls are almost in contact with each—other when a patient is in a relaxed condition in the dorsal position. The internal pressure along the vaginal channel may be close to zero.

During the vaginal probe 200 insertion, the vaginal probe finds an equilibrium between two opposite sides (anterior versus posterior) and the operator may advance the vaginal probe 200 to follow the insertion angle offering the least resistance along the vaginal canal. The vaginal probe insertion 200 may be completed in 10 seconds and the operator observes all acquired tactile images in real-time. The vaginal probe depth insertion may be defined by the pressure signals from the vaginal hiatus. Various measurements taken along the insertion path may be combined into a composed image 309, which demonstrates zones with increased pressure in both anterior and posterior compartments. Spatial pressure gradients calculated from the vaginal canal in the anterior and posterior directions, shown as white dotted lines in image 309, may also be placed over the image 309 to reveal distributions of tissue elasticity along anterior and posterior compartments. The tactile image 309 characterizes the vaginal tissue elasticity behind the vaginal walls at a distance comparable with the value of the tissue deformation (2-10 mm).

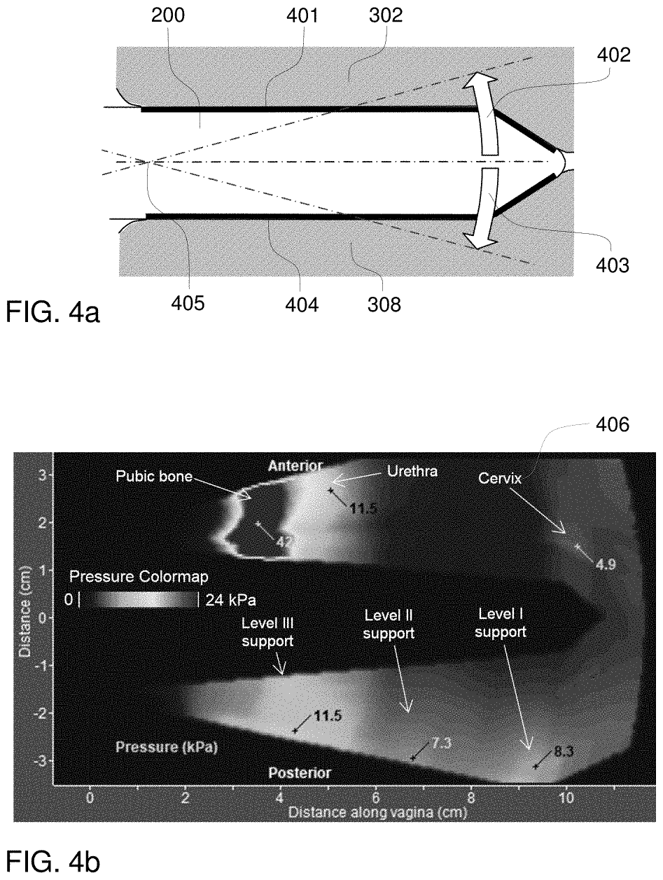

FIGS. 4a and 4b illustrates probe elevation (Test 2) and acquired tactile image for anterior 302 and posterior 308 vaginal compartments. The vaginal probe 200 elevation, as shown by arrows 402 and 403, allows the acquisition of pressure feedback from the pelvic floor structures at about 15-40 mm depth under significant tissue deformation (up to 45 mm) for the anterior and posterior vaginal compartments. The vaginal probe 200 may include an orientation sensor to measure an elevation angle, such that the pressure feedback acquired by pressure sensor arrays 401 and 404 from the contact with the vaginal walls may be mapped along the elevation angle as shown in tactile image 406. The up and down elevation of the vaginal probe 200 may be made relative to the hymen 405. In the anterior compartment, from left to right, tactile responses from the pelvic bone (pubic symphysis), the urethra, and the cervix may be observed in the image 406. In the posterior compartment, from left to right, tactile responses from Level III support, Level II support, and Level I support may be seen. Level III support includes perineum, Puborectal muscle, Level II support includes puboanal and pubovaginal muscles, and Level I support includes iliococcygeal muscle, levator plate, cardinal and uterosacral ligaments.

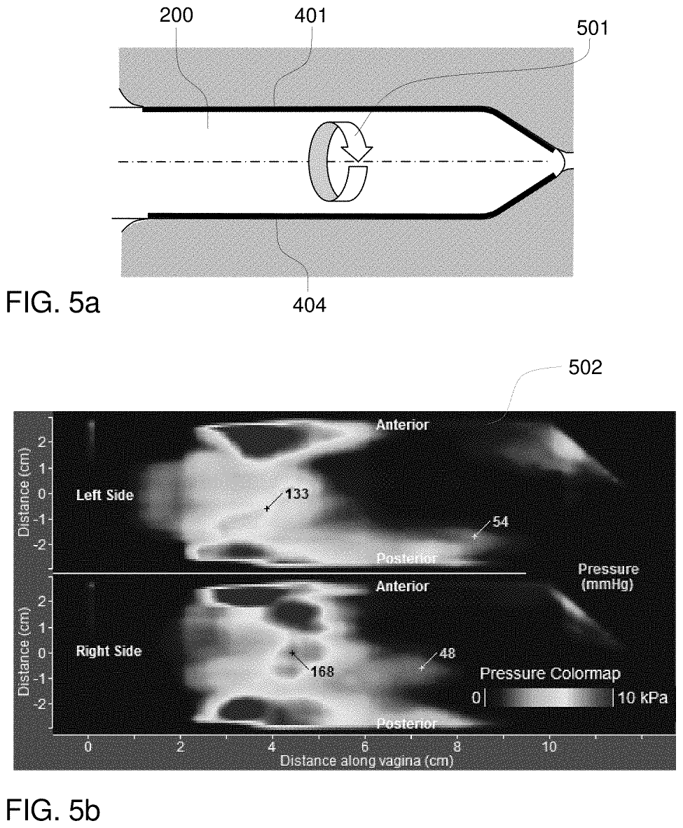

FIGS. 5a and 5b illustrate examination using vaginal probe rotation (Test 3) and acquired tactile image 502 for the left and right sides of the vagina. The vaginal probe 200 is rotated by 360 degrees as shown by arrow 501, allows acquisition of circumferential pressure feedback from the vaginal walls along the entire vagina. The vaginal probe 200 may be equipped with an orientation sensor to measure its rotational angle, such that the pressure feedback from tactile arrays 401 and 404 may be mapped along the rotation angle, as shown in FIG. 5—for one or both left and right sides. It may be useful that the vaginal probe 200 is rotated slowly for better control of the image quality on the computer display. The vaginal walls may be deformed up to 7 mm during the vaginal probe 200 rotation due to the difference in probe sizes in its orthogonal directions. Any local pressure peaks at the vaginal sides may be interpreted as stiff irregularities or lumps on the vaginal wall or behind it at a depth of about 0-7 mm. Asymmetry in pressure patterns on one side versus the other side conveys information about the asymmetry in pelvic floor structures behind the vaginal walls.

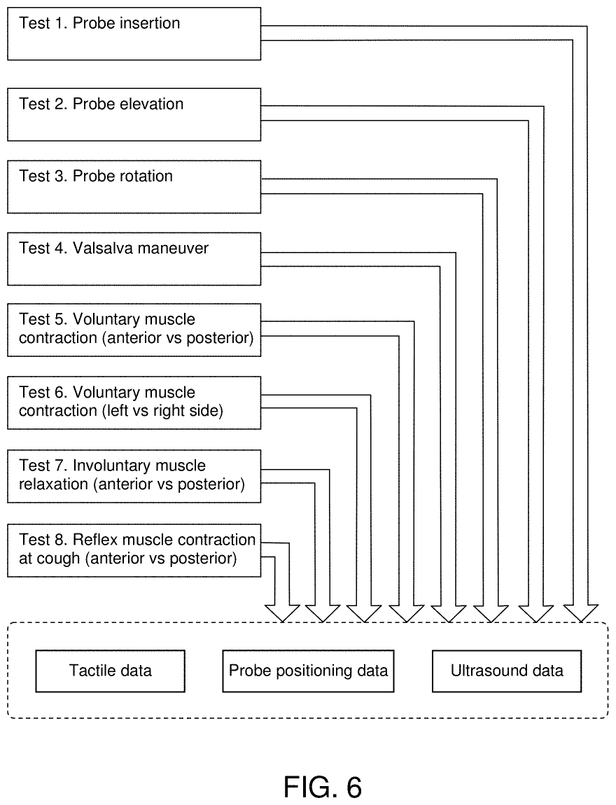

FIG. 6 presents an exemplary flow chart of tactile, ultrasound, and probe positioning data acquisition during a patient examination with a vaginal probe of the invention. The pelvic floor examination procedure may include up to 8 tests. These tests may be used to acquire the following information relevant to pelvic organ prolapse, urinary incontinence, pelvic pain, and tissue atrophy.

Test 1 (probe insertion): Tactile image and ultrasound images for vaginal anterior and posterior compartments along the entire vagina; tissue elasticity for the specific area(s)

Test 2 (probe elevation): Tactile image and ultrasound image for anterior and posterior compartments along the entire vagina; pelvic floor support

Test 3 (probe rotation): Circumferential tactile image of vaginal canal and a 3D ultrasound image of the pelvic floor; tissue elasticity for the specific area(s) of interest; structure anatomical sizes

Test 4 (Valsalva maneuver): Tactile responses along the entire vagina and ultrasound images for anterior and posterior compartments; static and contraction pressure patterns; muscle contractive mobility; structure and muscle identification

Test 5 (voluntary contraction): Tactile responses along the entire vagina and ultrasound images for anterior and posterior compartments; static and contraction pressure patterns; structure and muscle identification

Test 6 (voluntary contraction): Tactile responses along the entire vagina and ultrasound images for left and right sides; static and contraction pressure patterns; structure and muscle identification

Test 7 (involuntary relaxation): Pressure dynamic for pelvic muscle relaxation (weakening) along the entire vagina for anterior and posterior compartments; relaxation graphs (slope); structure identification

Test 8 (reflex contraction): Tactile responses along the entire vagina and ultrasound images for anterior and posterior compartments; static and contraction pressure patterns; muscle contractive mobility; structure and muscle identification

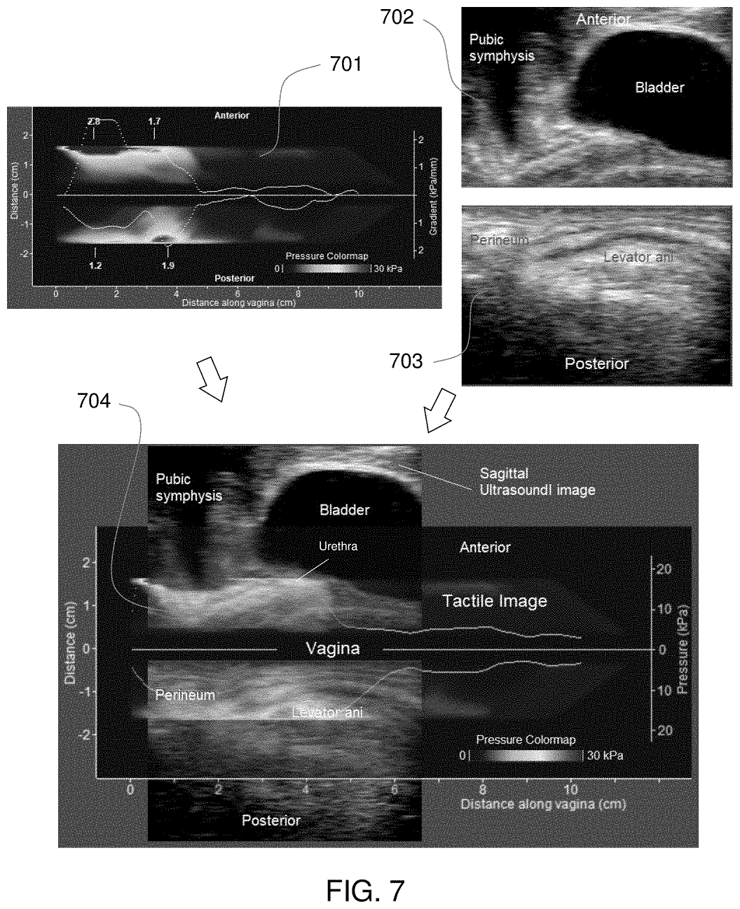

FIG. 7 illustrates a step of producing a tactile and ultrasound image fusion for Test 1 data (probe insertion), which allows elasticity assessment and broad characterization of the female pelvic floor using a single image. In this case, image fusion of critical structures for a 66 y.o. woman with normal pelvic floor conditions is shown. Specifically, tactile image 701 is seen overlaid or fused with anterior ultrasound image 702 and posterior ultrasound image 703. This fusion is based on known relative tactile sensors and ultrasound transducer array placement on the vaginal probe 200. The scale of both images may be the same for the correlation of key landmarks or position of sensors-transducers on the probe 200. The tactile image 701 may be made semitransparent to allow visualization of the ultrasound image features through the tactile image. This fusion provides identification of the pelvic structures which make contributions to the areas of increased elasticity in anterior and posterior compartments. In this specific example, the anterior elasticity peak of 1.7 kPa/mm (see image 701) is attributed to the urethra, the posterior elasticity peak of 1.2 kPa/mm (see image 701) is attributed to the perineum, and the posterior elasticity peak of 1.9 kPa/mm (see image 701) is attributed to the levator ani muscles. Generally, such image fusion allows identification and elasticity characterization of major pelvic structures.

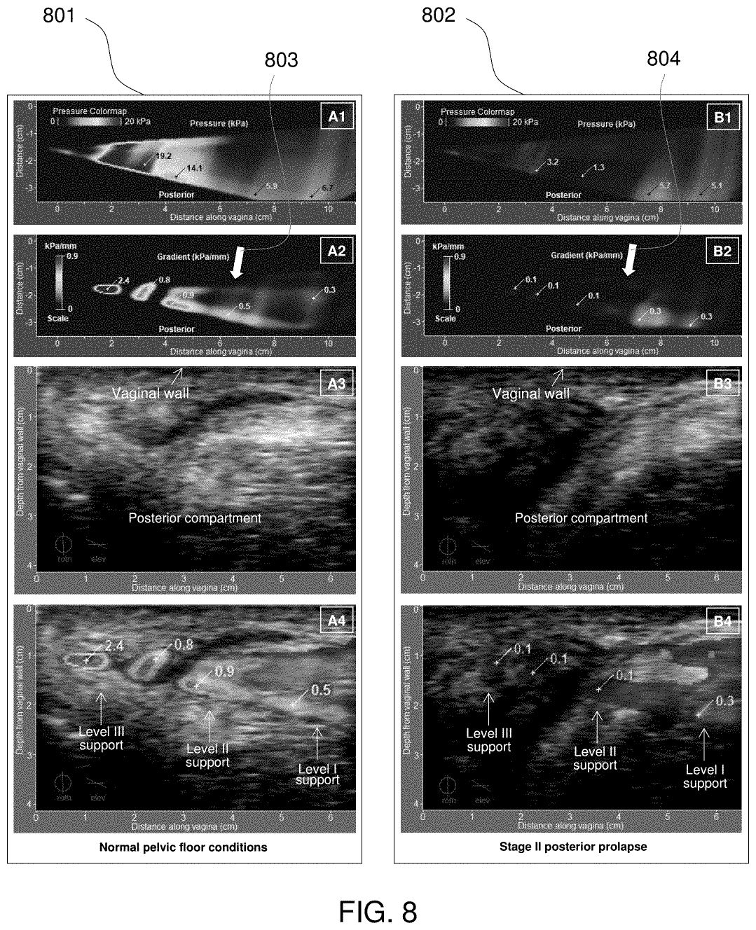

FIG. 8 illustrates another example of tactile and ultrasound image fusion for Test 2 data (probe elevation), which allows for a comprehensive assessment of pelvic floor support. A set of images 801 may be acquired and processed for a woman with normal pelvic floor conditions (left, A1-A4) and a set of images 802 is for a subject with pelvic organ prolapse stage II (right, B1-B4). In this test, the posterior vaginal wall may be deformed up to 45 mm during the vaginal probe elevation—see the tactile images in A1 and B1; so, that the pelvic floor support can be quantified at all Levels I-III. This is made possible because the deeper support structures contribute to the acquired pressure patterns on the vaginal wall at such deformations. Images shown in A2 and B2 may be gradient images calculated from the tactile images A1 and B2. Arrows 803 and 804 show the direction in which these spatial pressure gradients were calculated. Images A3 and B3 are the sagittal ultrasound images for the posterior vaginal compartment. And finally, image fusion for the gradient images with the ultrasound images is presented in A4 and B4. One may observe in A4 and B4 that the gradient images are placed at about 10 mm depth from the vaginal wall inside the ultrasound images and rotated about 5 degrees in a clockwise direction to make concordance of identified pelvic structures in ultrasound images with the gradient images which show the strength of the pelvic floor support.

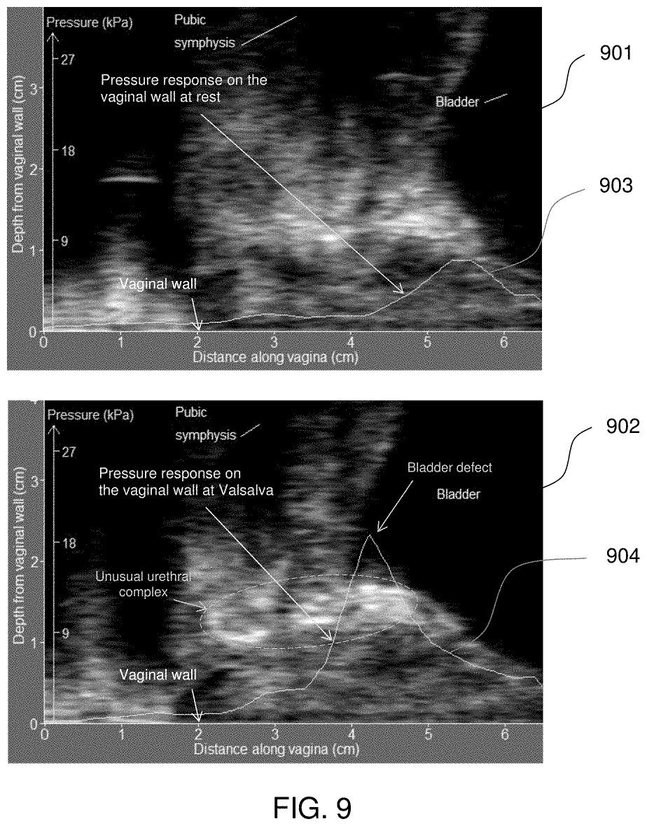

FIG. 9 illustrates a further example of the ultrasound image fusion with dynamic tactile patterns (tactile responses) for Test 4 data for a patient with anterior Stage III POP and overactive bladder at rest (left) and at Valsalva maneuver (right). The yellow line 903 along the anterior vaginal wall (bottom horizontal axis) is the pressure distribution at rest and yellow line 904 along the anterior vaginal wall (bottom horizontal axis) is the pressure distribution at Valsalva maneuver for the same probe position in the same patient. The fusion of the pressure response 904 with the ultrasound image 902 allows identification of the urethral complex and its biomechanical characterization, which includes the pressure peak change and displacement (mobility) at the Valsalva maneuver relative to the rest conditions. Unusual urethral appearance, the significant pressure peak change, and mobility are observed for this patient.

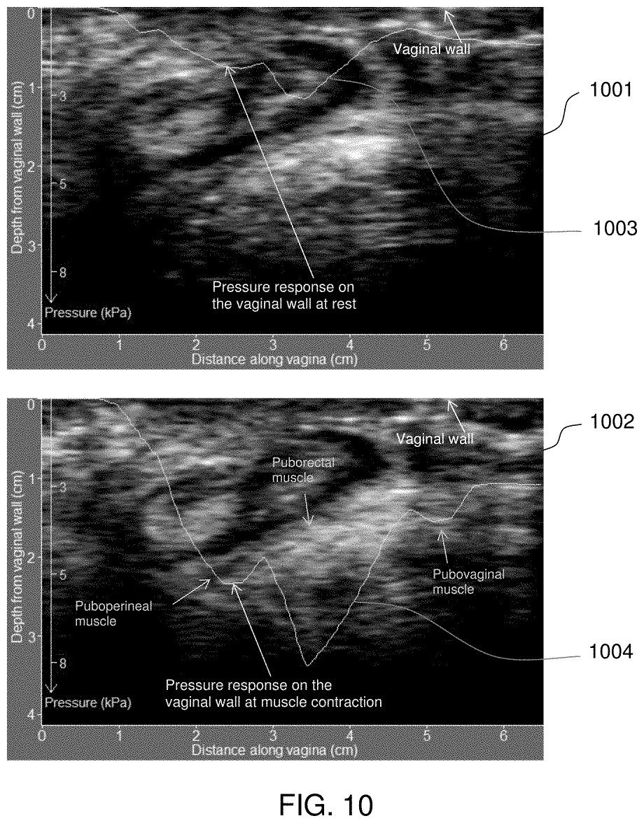

FIG. 10 illustrates a further yet example of the ultrasound image fusion with tactile responses for Test 5 data for a patient without posterior prolapse, but with uterine Stage II POP at rest (left) and at voluntary pelvic muscle contraction (right). The yellow line 1001 along the posterior vaginal wall (upper horizontal axis) is the pressure distribution at rest and yellow line 1002 along the posterior vaginal wall (upper horizontal axis) is the pressure distribution at voluntary muscle contraction for the same probe position in the same patient. The fusion of the dynamic tactile pattern of the pressure response 1002 with the ultrasound image 1004 allows identification of the pubopeerineal, puborectal, and pubovaginal muscle and their biomechanical characterization, which includes the pressure peak changes and anatomical changes at contraction relatively the rest conditions. An unusual weak contractive signal from the pubovaginal muscle is observed for this patient.

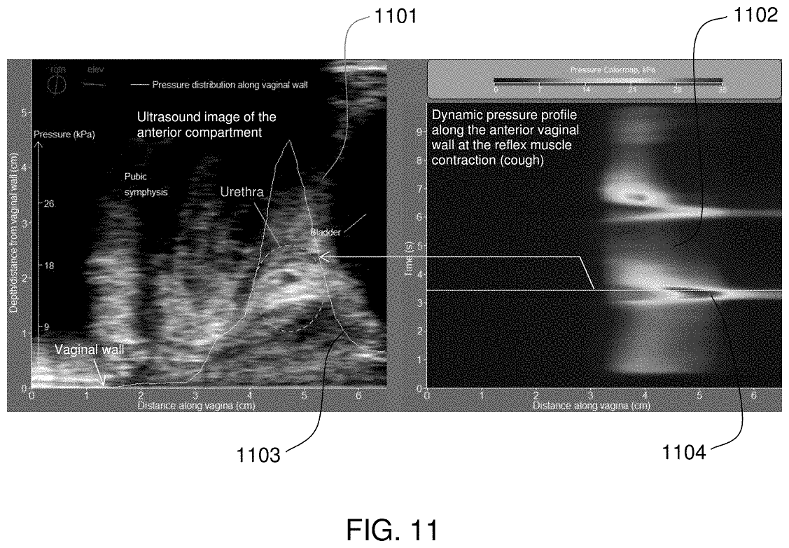

FIG. 11 illustrates another example of image fusion—in this case merging the ultrasound image 1001 with tactile response 1103 for Test 8 data for a patient with normal pelvic support (no prolapse), but with stress urinary incontinence and hyperactive urethra. The yellow line 1003 along the anterior vaginal wall (bottom horizontal axis) is the pressure distribution at reflex muscle contraction at cough. The right image 1102 is the dynamic pressure profile on the anterior vaginal wall to visualize the reflex pelvic muscle contraction 1104 at a cough where the vertical axis is the time and the horizontal axis is the pressure distribution along the anterior vaginal wall. Image fusion of the pressure response 1103 with the ultrasound image 1101 allows identification of the urethra, which includes the pressure peak value from the urethra at contraction and its anatomical appearance. The urethral mobility may be calculated by comparing the positioning of the urethra at rest with its positioning at contraction in image 1101. The urethral mobility may also be calculated from the dynamic image 1102 by analyzing the peak displacement 1104 along the vaginal wall. Significant urethral mobility is observed for this patient.

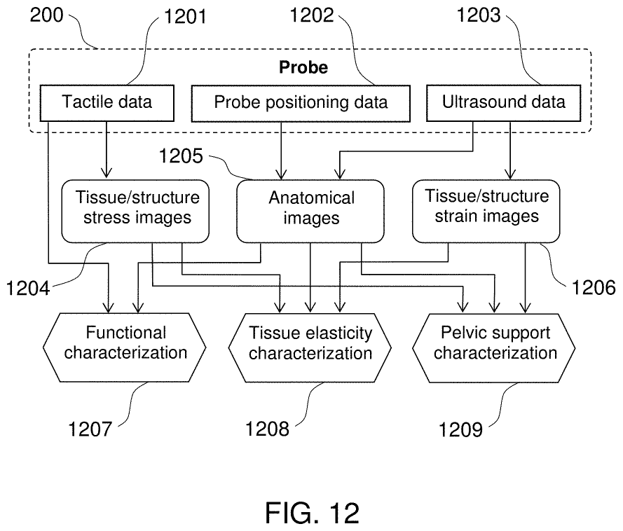

FIG. 12 is a flow chart of a method for performing vaginal tactile and ultrasound imaging according to the present invention. Tactile data 1201, probe positioning data 1202 and ultrasound data 1203 obtained during a patient examination with a vaginal probe 200 allow composing of (a) tissue and structure stress images 1204 from the tactile data, (b) anatomical images 1205 from probe positioning and ultrasound data, and (c) tissue and structures strain images 1206 from ultrasound strain data. Presenting image fusion as it is outlined in FIG. 12 allows for a more comprehensive pelvic functional characterization 1207, tissue elasticity characterization 1208, and pelvic support characterization 1209.

The pelvic floor anatomical image may be generated by a conventional ultrasound imaging approach. The tissue/structure stress images may be derived from the tactile images under tissue deformation (see tactile imaging definition above). The tissue/structure strain images may be generated by following a set of pixels with an image recognition technique within the ultrasound image for an area of interest during the tissue/structure deformation.

The muscle functional images may be generated from ultrasound dynamic images and dynamic tactile responses during pelvic muscle contraction. Further, all images may be segmented into pelvic structures and muscles (such as puborectalis, pubococcygeus, pubovaginal, puboperineal, levator plate, ilicoccygeal). Further yet, some or all of segmented structures may be quantitatively characterized based of digital data provided by elasticity images and muscle functional images.

Accurate probe motion tracking may allow for probe positioning data in real time within the image generated on the computer display. Imaging with a moving probe may increase the ultrasound as well as tactile image resolution.

Circumferential 3D ultrasound and tactile image formation in Test 3 (probe rotation) may be performed using probe orientation data from the motion tracking system.

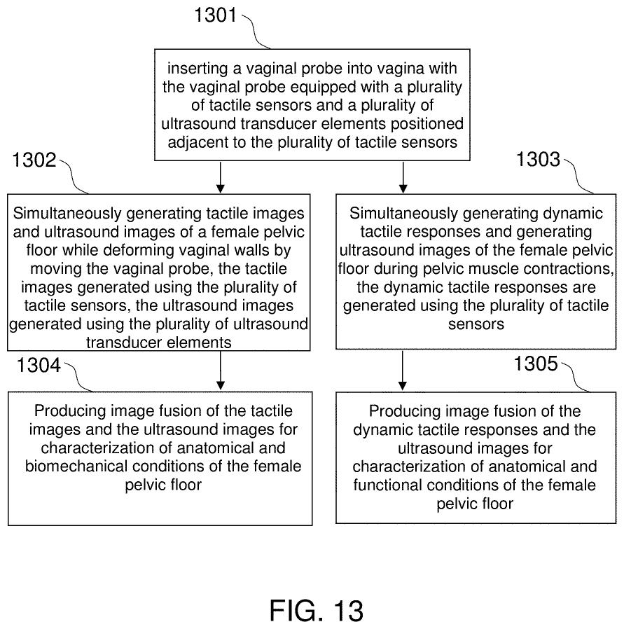

FIG. 13 is a flow chart for an embodiment of a method for vaginal tactile and ultrasound image fusion. A method for vaginal tactile and ultrasound image fusion may include the steps of:
(a) inserting a vaginal probe into vagina with the vaginal probe equipped with a plurality of tactile sensors and a plurality of ultrasound transducer elements positioned adjacent to the plurality of tactile sensors, 1301,
(b) simultaneously generating tactile images and ultrasound images of a female pelvic floor while deforming vaginal walls by moving the vaginal probe, the tactile images generated using the plurality of tactile sensors, the ultrasound images generated using the plurality of ultrasound transducer elements, 1302,
(c) simultaneously generating dynamic tactile responses and generating ultrasound images of the female pelvic floor during pelvic muscle contractions, the dynamic tactile responses are generated using the plurality of tactile sensors, 1303,
(d) producing image fusion of the tactile images and the ultrasound images generated in step (b) for characterization of anatomical and biomechanical conditions of the female pelvic floor, 1304, and
(e) producing image fusion of the dynamic tactile responses and the ultrasound images generated in step (c) for characterization of anatomical and functional conditions of the female pelvic floor.

Additional steps may include composing tissue elasticity images from the tactile images and the ultrasound images, composing muscle functional images from the tactile responses and the ultrasound dynamic images, and characterizing pelvic floor tissues and muscles as biomechanical elements based on the tissue elasticity images and the muscle functional images.

The vaginal wall deformations may be produced by vaginal probe insertion, by vaginal probe elevation, and by vaginal probe rotation in the vaginal canal. In embodiments, tissue deformation may also be caused by pelvic floor muscle contractions generated by voluntary and involuntary reflex.

The acquisition of tactile and ultrasound data may be performed from one or two opposing vaginal walls along the entire vaginal canal.

The step of composing tissue elasticity images may be conducted with the use of tissue stress data derived from the tactile images and tissue strain data derived from the ultrasound images.

The step of composing muscle functional images may be conducted with the use of tactile response data overlaid onto contracting muscles identified in the ultrasound dynamic images.

It is contemplated that any embodiment discussed in this specification can be implemented with respect to any method of the invention, and vice versa. It will be also understood that particular embodiments described herein are shown by way of illustration and not as limitations of the invention. The principal features of this invention can be employed in various embodiments without departing from the scope of the invention. Those skilled in the art will recognize or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12, 15, 20 or 25%.

All of the devices and/or methods disclosed and claimed herein can be made and executed without undue experimentation in light of the present disclosure. While the devices and methods of this invention have been described in terms of preferred embodiments, it will be apparent to those of skill in the art that variations may be applied to the devices and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A method for vaginal tactile and ultrasound image fusion, said method comprising the steps of:
a) inserting a vaginal probe into vagina, said vaginal probe equipped with a plurality of tactile sensors and a plurality of ultrasound transducer elements positioned adjacent to said plurality of tactile sensors,
b) simultaneously generating tactile images and ultrasound images of a female pelvic floor while deforming vaginal walls by moving said vaginal probe, said tactile images generated using said plurality of tactile sensors, said ultrasound images generated using said plurality of ultrasound transducer elements,
c) simultaneously generating dynamic tactile responses and generating ultrasound images of the female pelvic floor during pelvic muscle contractions, said dynamic tactile responses are generated using said plurality of tactile sensors,
d) producing image fusion of said tactile images and said ultrasound images generated in step (b) for characterization of anatomical and biomechanical conditions of the female pelvic floor, and
e) producing image fusion of said dynamic tactile responses and said ultrasound images generated in step (c) for characterization of anatomical and functional conditions of the female pelvic floor.

2. The method as in claim 1, wherein in said step (a) said vaginal probe comprising a vaginal probe housing equipped with two tactile sensor arrays located on opposite sides thereof, said vaginal probe further comprising a first ultrasound transducer array located adjacent one of said two tactile sensor arrays.

3. The method as in claim 2, wherein in said step (a) said vaginal probe comprising a vaginal probe housing equipped with two tactile sensor arrays located on opposite sides thereof, said vaginal probe further comprising a second ultrasound transducer arrays located adjacent the other one of said two tactile sensor arrays.

4. The method as in claim 1, wherein in said step (a) said vaginal probe further comprising an orientation sensor configured for providing rotational and elevation angles of said vaginal probe.

5. The method as in claim 1, wherein in said step (a) said vaginal probe is equipped with a tapered tip configured for atraumatic insertion, said step (b) further comprising a step of moving said vaginal probe along the vaginal canal to deform anterior and posterior vaginal walls, said step (d) further comprising a step of producing image fusion of said tactile images from anterior and posterior vaginal walls and said ultrasound images for anterior and posterior vaginal compartments, whereby said image fusion is performed to identify and characterize elasticity as a stress-strain ratio for at least one of urethra, perineum, and levator ani.

6. The method as in claim 1, wherein in said step (a) said vaginal probe is equipped with a tapered tip configured for atraumatic insertion, said step (b) further comprising a step of elevation of said vaginal probe to deform posterior vaginal compartment and a step of generating a calculated gradient images from said tactile images, said step (d) further comprising a step of producing image fusion of said calculated gradient images and said ultrasound images for said posterior vaginal compartment, whereby said image fusion is performed to identify pelvic floor support components and characterize strength of pelvic floor support.

7. The method as in claim 1, wherein in said step (a) said vaginal probe is equipped with a tapered tip configured for atraumatic insertion, said step (b) further comprising a step of deforming said vaginal walls by rotating said vaginal probe, said step (d) further comprising a step of producing image fusion of said dynamic tactile responses and said ultrasound images, whereby said image fusion is performed to identify and characterize tissue elasticity as a stress-strain ratio for area of interest.

8. The method as in claim 1, wherein said step (c) further comprising a step of involuntary pelvic muscle contraction during Valsalva maneuver, wherein said simultaneously recorded dynamic tactile responses from vaginal walls and ultrasound images are fused together in step (e) to identify and characterize urethra, perineal muscle and levator ani muscles strength and mobility.

9. The method as in claim 1, wherein said step (c) further comprising a step of a voluntary pelvic muscle contraction, wherein said step (e) further comprising a step of producing a fusion image of simultaneously recorded dynamic tactile responses from posterior compartment and ultrasound images for posterior compartment to identify and characterize perineal muscles and levator ani muscles contractile strength.

10. The method as in claim 10, wherein said step (e) further comprising a step of producing an fusion image of said simultaneously recorded dynamic tactile responses from left and right sides of vagina and ultrasound images for left and right sides of vagina to identify and characterize pubovaginal and puboanal muscles contractive strength.

11. The method as in claim 1, wherein said step (c) further comprising a step of reflex pelvic muscle contraction at cough, said step (e) further comprising a step of producing an image fusion of said simultaneously recorded tactile responses from anterior and posterior vaginal walls and ultrasound images for pelvic floor structures behind said vaginal walls to identify and characterize contractile strength and mobility of at least one of urethral muscle, perineal muscles and levator ani muscles.

12. The method as in claim 5, wherein said step (b) further comprising a step of calculated said strain from said ultrasound images.

13. The method as in claim 8, wherein said step (c) further comprising a step of calculating said muscle mobility from said ultrasound images.
